(19) United States
(12) Patent Application Publication
(10) Pub. No.: US 2014/0296746 A1
(43) Pub. Date: Oct. 2, 2014

(54) URINE FLOW MEASURING APPARATUS

(71) Applicants: Michael Whitaker, Newcastle-upon-Tyne (GB); Michael Drinnan, Alnwick (GB); Alison Bray, Newcastle-upon-Tyne (GB); Clive Griffiths, Newcastle-upon-Tyne (GB); Robert Beckwith, Ashington (GB)

(72) Inventors: Michael Whitaker, Newcastle-upon-Tyne (GB); Michael Drinnan, Alnwick (GB); Alison Bray, Newcastle-upon-Tyne (GB); Clive Griffiths, Newcastle-upon-Tyne (GB); Robert Beckwith, Ashington (GB)

(73) Assignee: Newcastle-upon-Tyne Hospitals NHS Foundation Trust, Newcastle-upon-Tyne (GB)

(21) Appl. No.: 14/238,089

(22) PCT Filed: Aug. 9, 2012
(86) PCT No.: PCT/GB2012/051941

§ 371 Date: Mar. 26, 2014

(30) Foreign Application Priority Data
Aug. 10, 2011 (GB) 1113749.4

(51) Int. Cl. A61B 5/20

(52) U.S. Cl. CPC A61B 5/208
USPC 600/584

(57) ABSTRACT

A urine flow measuring apparatus that includes a container for receiving urine, measuring means for measuring the weight and/or volume of urine received by the container, and memory means for recording the weight and/or volume measured by the measuring means. Through a controller the measuring means: continually measures the weight and/or volume of the container at regular sampling intervals; and at each sampling interval, identifies if a positive change in the weight and/or volume of the container has occurred and then compares results within a data set, the data set including a result from the current sampling interval and results from all the sampling intervals during a given period. If a number of sampling intervals indicating a positive change in the weight and/or volume is above a certain threshold, the memory means is instructed to begin recording data, else the memory means is instructed to cease recording data.

### FIELD OF THE INVENTION

The invention relates to urine flow measuring apparatus and in particular to portable urine flow measuring apparatus that automatically records urine flow data.

### BACKGROUND OF THE INVENTION

Lower urinary tract symptoms (LUTS) are a common problem affecting around 30% of men aged over 50. LUTS include weak urine flow, the need to urinate frequently and the need to get up at night to urinate. These symptoms often have a detrimental effect on the sufferer's quality of life.

A major cause of male LUTS is non-cancerous enlargement of the prostate which results in the prostate obstructing the outlet of the bladder (bladder outlet obstruction, BOO). This condition can be relieved by drugs or by surgery to remove part of the prostate.

A measurement of the maximum, or peak, rate of urine flow is useful as a clinical indicator of urodynamic obstruction. A low maximum urinary flow rate of less than 15 ml/s is a key indication of likely obstruction and indicates that drug or surgical treatment may be beneficial.

Uroflowmetry is the test that measures urinary flow rate, it is the most useful non-invasive technique for the objective assessment of LUTS.

The test is currently typically carried out in a clinic using expensive electronic equipment, such as a weight transducer flow meter, into which the patient must urinate. This is normally followed by an ultrasound scan of the bladder to measure the amount of residual urine in the bladder. The equipment measures the peak flow, voided volume and flow trace with considerable precision. According to good practice the patient should perform two or three voids in the clinic, however there is often not sufficient time in the clinic to achieve this.

In addition to measuring the maximum urinary flow rate in the clinic the patient is often required to complete a ‘voiding diary’ at home for a number of days. Completion of a ‘voiding diary’ requires the patient to urinate into a jug and to record the volume, time and date of each voiding event. The ‘voiding diary’ helps the medical practitioner diagnose the cause of the patient's symptoms.

Problems with the current methodology are that a particular patient's urine flow rate is inherently variable according to the time of day, the amount of fluid consumed and even the availability of the nearest toilet. Therefore, despite the precision of readings obtained by expensive clinic equipment, a single or even two or three measurements of flow recorded in hospital are likely to be unrepresentative of the patient's average flow. Completion of the voiding diary is also important in diagnosing the patient's condition, however measuring each void by hand and remembering to record all the results can be burdensome for patients who may have difficulty keeping a coherent record.

Measurement of urine flow rate in a hospital clinic may also be unpleasant for the patient; sometimes they are required to arrive at the clinic with a full bladder, which in itself is a challenging task for a patient suffering from urinary problems. In order to take repeated measurements the patient must typically attend the clinic for a whole morning or afternoon, spending most of the time in the waiting room drinking copiously in order to repeatedly fill his bladder. The nature of the clinic flow test may prevent the recordal of a representative measurement of urinary flow rate. The clinic apparatus also requires specially trained staff to operate it. The unnatural clinic environment can affect the accuracy of the test result obtained.

US2010/0064797 describes a portable urine flow measuring apparatus comprising a container that receives urine and a scale. The scale may be held by the user during voiding of urine into the container. To use the device the user must first switch it on, then void into the container. The scale measures the weight of the container multiple times at given time intervals. After the device is switched on the measurement data may be recorded onto a memory card in the device. The device is then switched off by the user. This device requires interaction with the user in order to record the voiding data, and this could prove stressful for a user when urgent voiding is required. Failure to activate the device to record the data would mean that the voiding diary is not complete.

It would be desirable to provide an improved portable urine flow measurement apparatus that can be used by the patient to produce a representative measurement and record of urinary flow rate.

### SUMMARY OF THE INVENTION

One aspect of the invention provides a urine flow measuring apparatus comprising:
a container having an opening for receiving urine;
measuring means for measuring the weight and/or volume of urine received by the container;
memory means for recording the weight and/or volume of urine measured by the measuring means; and
a controller;

and wherein the controller automatically detects a positive change in weight and/or volume of the container measured by the measuring means and instructs the memory means to record the weight and/or volume of the container at pre-determined time intervals until no further change in weight and/or volume is detected.

Advantageously, the controller automatically detects and/or records only valid positive changes in weight and/or volume.

The controller may automatically detect and/or record positive changes in weight and/or volume above a threshold magnitude.

The controller may include a low pass filter. A low pass filter reduces noise and high frequency changes which may occur if the container is knocked.

The controller may automatically detect and/or record all positive changes in weight and/or volume, with invalid records being removed in a post processing step. The controller may automatically detect and/or record all positive changes in weight and/or volume above a threshold magnitude. The post processing step may include the step of applying a low pass filter to the recorded data.

Preferably, the controller continually measures the weight and/or volume of the container at regular sampling time intervals. Preferably, at each sampling time interval the controller identifies if a positive change in the weight and/or volume of the container has occurred and then compares the results from a data set comprising the result from the current sample and results from all the sampling intervals during a given period immediately prior to the current sample. If the number of sampling intervals which indicate a positive change in the weight and/or volume of the container is above a certain threshold value the controller instructs the memory means to begin recording of data. Preferably the controller further instructs the memory means the record the data from the sampling intervals during the given period immediately prior to the current sample. Preferably, if the number of sampling events which indicate a positive change in the weight and/or volume of the container is below a certain threshold value the controller instructs the memory means to cease recording of data.

Preferably the controller continues to measure the weight and/or volume of the container at regular sampling time intervals even when recording of data has ceased.

By comparing data results from a window of activity the apparatus is able to detect and record real urine flow results, and to reject and therefore not record transient apparent changes in weight caused, for example, by knocking the container.

The sampling time intervals are preferably at most one second apart. More preferably, the sampling time intervals are at most 0.25 seconds apart. Still more preferably, the sampling time intervals are at most 0.125 seconds apart.

Preferably the apparatus draws less power when the memory means is not recording data.

Preferably the measuring means measures the weight of urine received by the container and the controller calculates the volume of urine received by the container. The weight of urine received by the container is proportional to the volume of urine received the container. Preferably the controller is calibrated to calculate the volume of the urine and the volume of urine is recorded by the memory means.

The controller may be a micro-processor.

Preferably the memory means automatically records the duration of each urination event and the time between urination events. Preferably, the controller automatically instructs the memory means to record the relative time associated with each change in weight and/or volume of the container.

The memory means for recording the weight and/or volume of urine measured by the measuring means may include a memory chip or a removable memory card.

Weight and/or volume data recorded on the memory means may be transferred to a computer. Computer software may then be used to calculate the rate of change of volume, or the urine flow rate.

The data may be transferred to a computer from the measuring means via USB connection or via Bluetooth or via memory card reader.

Preferably the controller instructs the memory means to record the weight and/or volume of the container at least once every second after a positive change in weight and/or volume has been detected. More preferably, the controller instructs the memory means to record the weight and/or volume of the container at least four times every second. Still more preferably, the controller instructs the memory means to record the weight and/or volume of the container at least eight times every second.

Preferably the apparatus further comprises means for securely engaging the container with the measuring means. This prevents the container from moving or tipping during use.

The means for securely engaging the container with the measuring means may comprise a recess in the base of the container, the recess being shaped to correspond with the shape of the measuring means.

Preferably the opening of the container is funnel shaped. This makes the direction of urine into the container easier for the user.

The urine flow measuring apparatus of the invention provides a convenient, easy to use and reliable means for collecting urine flow measurements from patients in the privacy of their own home.

### BRIEF DESCRIPTION OF THE DRAWINGS

In the drawings, which illustrate preferred embodiments of the invention by way of example:

FIG. 1 illustrates a urine flow measuring apparatus according to an embodiment of the invention;

FIG. 2 illustrates the base unit of the urine flow measuring apparatus of FIG. 1, viewed from above;

FIG. 3 illustrates an underneath view of the base unit of FIG. 2;

FIG. 4 illustrates a side view of the apparatus of FIG. 1.

### DETAILED DESCRIPTION OF THE PREFERRED EMBODIMENTS

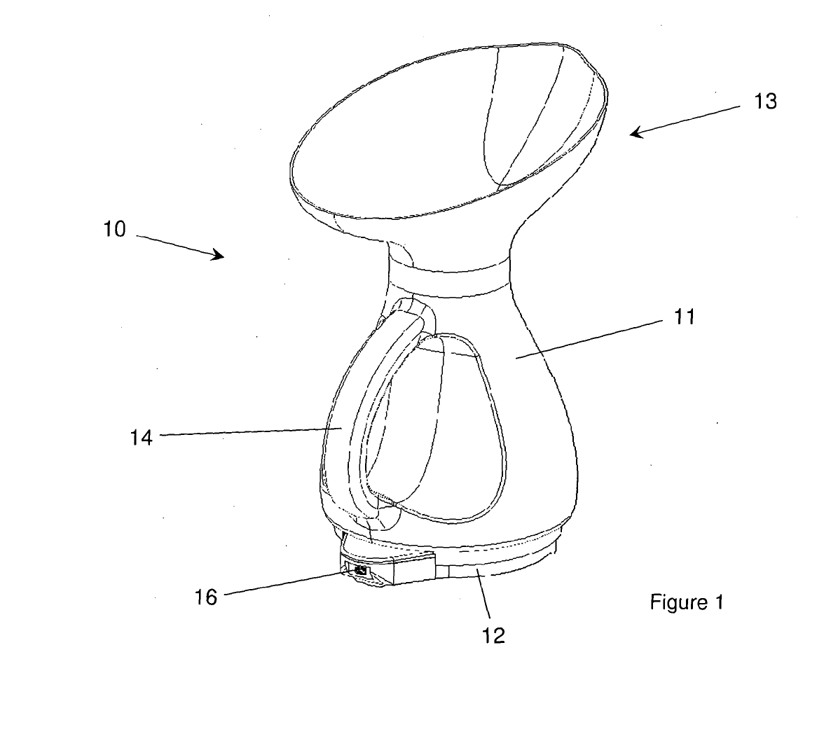

A urine flow measuring apparatus according to an embodiment of the invention is shown generally at 10 in FIG. 1. The apparatus 1 includes a jug shaped container 11 that receives urine and a base unit 12 that includes measuring means for measuring the weight and/or volume of the urine received by the container 11. To use the apparatus the container 11 is placed on top of the base unit 12. Preferably the container 11 has a funnel shaped opening portion 13 through which urine voided by an individual is received. The funnel shaped opening portion 13 makes it easier for a user to direct the urine into the container 11. The container 11 is preferably made from plastic, but could be made from any material suitable for containing urine. The container 11 is preferably provided with a handle portion 14. The handle portion 14 makes it easier for the user to transport the container 11 for disposal of the urine and subsequent cleaning.

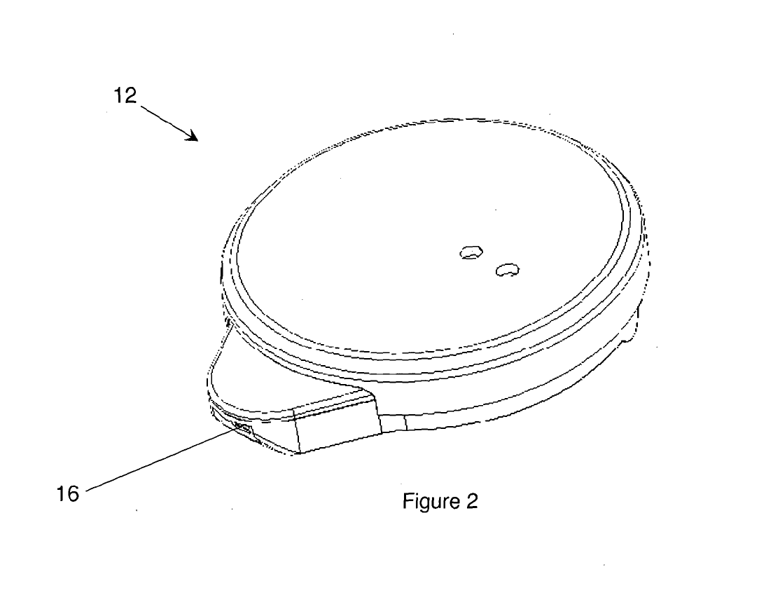

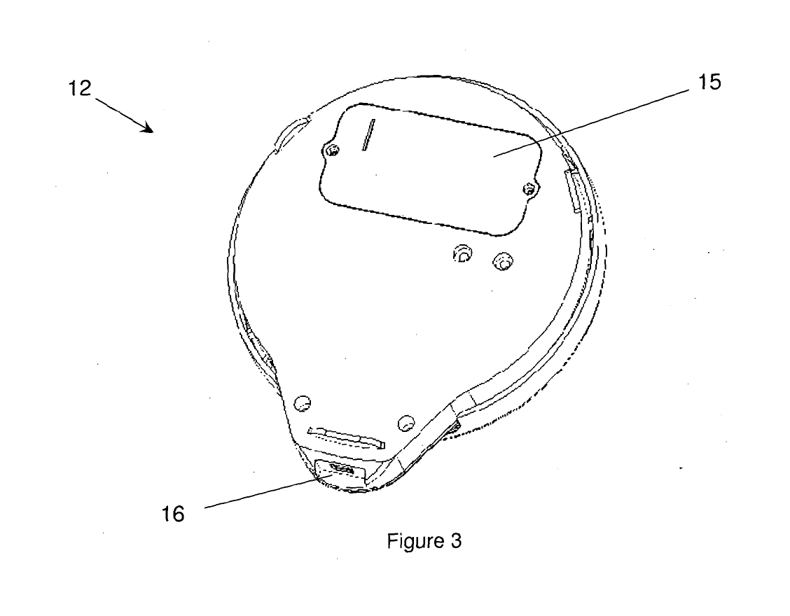

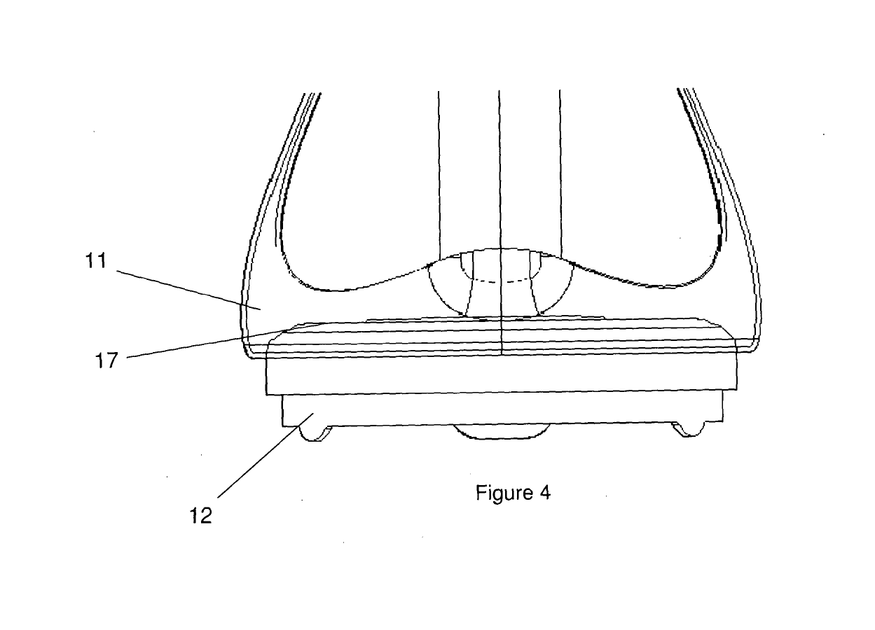

FIG. 2 illustrates the base unit 12 in more detail. Preferably the base unit 12 is powered by batteries located inside the battery compartment 15 on the underside of the base unit 12 as shown in FIG. 3, but could be powered by another suitable source of electrical energy.

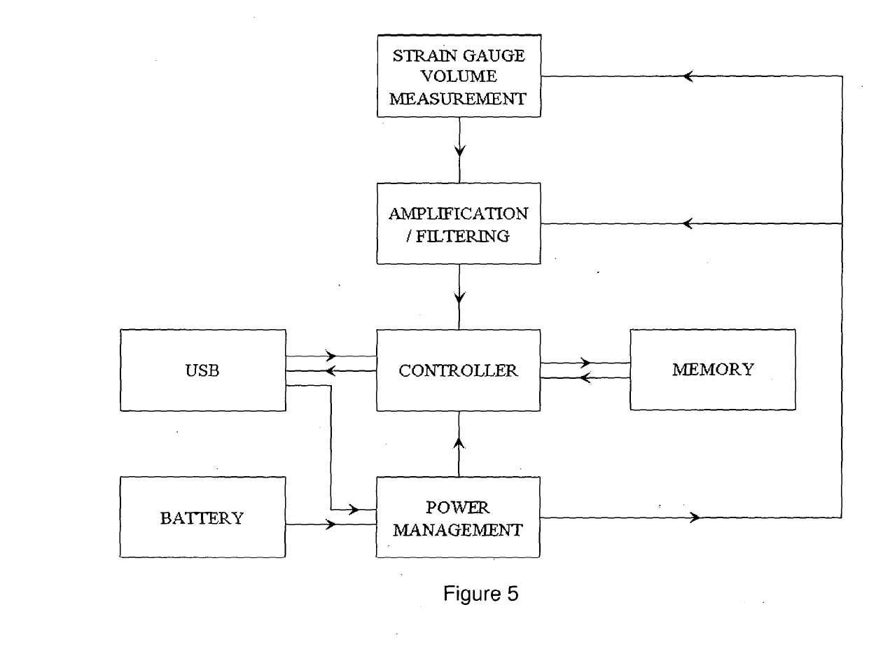

Preferably, as shown in the illustrated embodiment in FIG. 4, the base of the jug container 11 has a recessed portion 17 shaped such that it fits over the base unit 12. This means that the container 11 and the base unit 12 fit together securely making it less likely that the jug will move or tip whilst it is being used. As shown in the drawing the edge of the container 11 overhangs the base unit 12, protecting the base unit 12 from any splashes that may occur during use.

The apparatus is used with the jug container 11 positioned on top of the base unit 12. The base unit 12 includes an electronic component called a load beam. As the weight applied to the base unit 12 changes the resistance of the load beam changes. A Wheatstone bridge converts the change in resistance to a change in voltage, which changes proportionally to the volume of voided urine received by the container. A controller is located inside the base unit 12. In this example the controller is calibrated to convert the voltage measured to a volume measurement and volume measurements are recorded onto memory located inside the base unit 12.

The controller periodically checks for changes in the weight of the container 11. When a positive change in weight is detected the controller automatically instructs the recording of volume data to the memory chip. Volume data is recorded at pre-determined time intervals until no further change in weight is detected, indicating that voiding has finished. In this example the volume of urine is recorded every 0.125 seconds, or eight times every second. The apparatus is calibrated to take into account the weight of the container 11 when calculating the volume of urine received.

In a preferred embodiment the controller continually measures the weight or volume of the container 11 at pre-determined sampling time intervals. The result from the current sample is then compared with the results of samples taken during a period of time immediately preceding the current sample. For example the weight and/or volume of the container 11 may be measured every 0.125 seconds, and the results compared with the results from sampling events measured during the previous 5 seconds, giving a data set of approximately 40 individual results. In this embodiment of the invention each sample is identified as to whether it relates to a positive change in weight and/or volume of the container 11. If the number of sampling intervals within the data set which indicate a positive change in the weight and/or volume of the container 11 is above a certain threshold value the controller instructs the memory chip to begin recording of data. Preferably the 5 seconds of data from the data set immediately preceding the current sample is also recorded to the memory chip. If the number of sampling events indicating a positive change in the weight and/or volume of the container is below a certain threshold value the controller instructs the memory chip to cease recording of data.

In the illustrated embodiment an internal memory chip for recording information relating to voiding events is located inside the base unit 12. To retrieve the information from the chip the base unit 12 is plugged into a computer via a USB port 16 on the base unit 12 and the data is then downloaded to the computer. The base unit 12 may be brought into the clinic by the patient and the data then downloaded by the clinician. Alternatively, the patient may connect the base unit 12 to their own computer and the data may then be sent to the clinician electronically.

Alternatively, a removable memory card may be located inside the base unit 12 for recording information relating to voiding events. The patient may then remove the memory card from the apparatus in order to send it back to the clinician, or may download the data from the memory card onto their own computer and the data may then be sent to the clinician electronically.

Preferably the memory card or the internal memory chip records the date and time (either the actual time or the time relative to the last voiding event) of any voiding event, along with the weight or volume of the voided urine at pre-determined time intervals. The controller ensures that any change in weight of the container 11 is recorded. There is no requirement for the user to switch the apparatus 1 on in order to record the voiding data. When not recording, the apparatus 1 is in a standby mode in which the clock continues to run and the apparatus is ready to record any change in weight of the container.

Once the weight and/or volume and time data have been downloaded to a computer the urine flow rate may be calculated using computer software.

Typically the memory chip of the apparatus can accommodate around 1000 minutes' worth of data which is more than sufficient to record all of an individual's voiding events during a two week period. A two week voiding diary would typically comprise up to 100 minutes of data.

1. A urine flow measuring apparatus comprising:
a container having an opening for receiving urine;
measuring means for measuring at least one of a weight and a volume of urine received by the container; and
memory means for recording the at least one of the weight and volume of urine measured by the measuring means;
wherein the measuring means comprises a controller and through the controller the measuring means:
continually measures the at least one of the weight and volume of the container at regular sampling time intervals; and
at each sampling time interval, identifies if a positive change in at least one of the weight and volume of the container has occurred and then compares results from a data set, the data set comprising a result from the current sampling time interval and results from all the sampling time intervals during a given period immediately prior to the current sampling time interval;
wherein if a number of sampling time intervals from the data set which indicate a positive change in at least one of the weight and volume of the container is above a certain threshold value, the memory means is instructed to begin recording of data; and
wherein if the number of sampling time intervals from the data set which indicate a positive change in at least one of the weight and volume of the container is below a certain threshold value, the memory means is instructed to cease recording of data.

2. A urine flow measuring apparatus according to claim 1, wherein the measuring means measures the weight of urine received by the container and the controller calculates the volume of urine received by the container.

3. A urine flow measuring apparatus according to claim 1, wherein the controller automatically instructs the memory means to record a relative time associated with each change in at least one of the weight and volume of the container.

4. A urine flow measuring apparatus according to claim 1, wherein the memory means includes a memory chip or a removable memory card.

5. A urine flow measuring apparatus according to claim 1, wherein the data recorded on the memory means is transferrable to a computer.

6. A urine flow measuring apparatus according to claim 1, wherein the pre-determined time interval for recordal of the at least one of the weight and volume of the container is at least once every second.

7. A urine flow measuring apparatus according to claim 1, wherein the pre-determined time interval for recordal of the at least one of the weight and volume of the container is at least four times every second.

8. A urine flow measuring apparatus according to claim 1, wherein the pre-determined time interval for recordal of the at least one of the weight and volume of the container is at least eight times every second.

9. (canceled)

10. A urine flow measuring apparatus according to claim 1, wherein the controller further instructs the memory means to record the data from the sampling time intervals during the given period immediately prior to the current sample.

11. A urine flow measuring apparatus according to claim 1, wherein the sampling time intervals are at most one second apart.

12. A urine flow measuring apparatus according to claim 1, wherein the sampling time intervals are at most 0.25 seconds apart.

13. A urine flow measuring apparatus according to claim 1, wherein the sampling time intervals are at most 0.125 seconds apart.

14. A urine flow measuring apparatus according to claim 1, further comprising means for securely engaging the container with the measuring means.

15. A urine flow measuring apparatus according to claim 14, wherein the container comprises a base and the means for securely engaging the container with the measuring means comprises a recess in the base of the container, the recess being shaped to correspond with the shape of the measuring means.

16. (canceled)
